(19) United States
(12) Patent Application Publication
(10) Pub. No.: US 2017/0189166 A1
(43) Pub. Date: Jul. 6, 2017

(54) CORNEAL IMPLANTS AND METHODS OF MANUFACTURING

(71) Applicant: REVISION OPTICS, INC., Lake Forest, CA (US)

(72) Inventor: Keith HOLLIDAY, Lake Forest, CA (US)

(21) Appl. No.: 15/313,297

(22) PCT Filed: May 27, 2015
(86) PCT No.: PCT/US15/32656

§ 371 Date: Nov. 22, 2016

Related U.S. Application Data
(60) Provisional application No. 62/003,418, filed on May 27, 2014.

(51) Int. Cl. A61F 2/14

(52) U.S. Cl. CPC A61F 2/142; A61F 2/1451; A61F 2/1453; A61F 2/148; A61F 2/14; A61F 2/145; A61F 2002/1699

(57) ABSTRACT

Corneal implants that have an implant body comprising manufactured corneal tissue. Methods of manufacturing corneal implant that include manufacturing a volume of corneal tissue. Corneal implants that have an implant body made from cornea tissue removed from a living subject.

### CROSS REFERENCE TO RELATED APPLICATIONS

This application claims priority to U.S. Prov. App. No. 62/003,418, filed May 27, 2014, which is incorporated by reference herein.

This application is related to and incorporates by reference herein the disclosures of the following: U.S. Pat. No. 8,057,541, issued Nov. 15, 2011; U.S. Pub. No 2008/0262610, published Oct. 23, 2008; U.S. Pub. No 2009/0198325; and U.S. Pub. No. 2011/0218623.

### INCORPORATION BY REFERENCE

All publications and patent applications mentioned in this specification are herein incorporated by reference to the same extent as if each individual publication or patent application was specifically and individually indicated to be incorporated by reference.

### BACKGROUND

Human corneal tissue from donors can be used for transplant of entire corneas or portions of a cornea. The technology that allows this procedure to be performed has been available for several decades and is well-established.

It has recently been reported that it has become possible to manufacture corneal tissue using human keratocytes in vitro. This material can, for instance, be produced on a template that responds to enzymes. (Gouveia R., et al., (2014). Fabrication of a Bio-Prosthetic Cornea from Human Keratocytes [Abstract], which is incorporated by reference herein). The growth process can be ended when the keratocytes are triggered to degrade the template using secreted enzymes. Such tissue can be produced with a substantially similar structure to human corneal tissue so that the extracellular matrix will be composed of aligned collagen fibrils and proteoglycans that are deposited in lamellae. Gouveia's template was made from self-assembling peptide amphiphile (PA) molecules composed of a hexadecyl lipid chain attached to a peptide headgroup containing a metalloprotease (MMP)-responsive sequence followed by the integrin-binding motif Arg-Gly-Asp-Ser. Keratocytes were grown on these templates in serum-free media for up to 90 days, and the resulting tissue was analysed for stromal-specific ECM markers at transcriptional and protein levels. Retinoic acid (RA) at 1×10⁻⁵ M was added to culture media during growth to inhibit MMP expression from keratocytes, and then removed to elicit PA template degradation and tissue self release. Gouveia successfully fabricated human corneal tissue in vitro solely by culturing human donor-isolated keratocytes on a smart template. This template promoted integrin-mediated adhesion, and keratocyte proliferation and stratification. The addition of RA to serum-free media further enhanced these effects, and significantly increased the production of keratocyte markers such as collagen type-I, keratocan, lumican, and ALDH1. Furthermore, RA reduced MMP activity from culture supernatant to less than 5% of control, thus ensuring the integrity of the PA template throughout the entire period in culture and allowing the formation of structurally coherent, ECM-enriched, robust corneal tissue. By resuming MMP expression after RA removal, they were able to control template degradation and promote tissue self-release, minimizing damage from manipulation. Gouveia envisions applications in regenerative medicine.

Altering the refractive properties of the cornea can correct optical imperfections of the eye's focusing apparatus. LASIK is a procedure that removes tissue from the cornea by producing a keratotomy flap, lifting the flap and then ablating a predetermined quantity of tissue from the flap bed. When the flap is replaced on the bed the surface shape of the cornea has been amended and the optical power of the cornea has been changed. Other procedures amend corneal power in similar ways.

Keratophakia refers to a process in which artificial tissue is added to the cornea in a manner that has the same result as LASIK though using an additive rather than subtractive method. For instance, a meniscus-shaped piece of material that is larger than the pupil can be positioned within the cornea to correct hyperopia by increasing the power of the cornea. Similarly a smaller meniscus-shaped piece of tissue that is substantially smaller than the pupil can be introduced into the cornea, at least partially aligned with the pupil to produce an amended corneal power that varies across the region of the inlay. Such a method can be used to correct presbyopia, examples of which can be found in U.S. Pat. No. 8,057,541, issued Nov. 15, 2011; U.S. Pub. No 2008/0262610, published Oct. 23, 2008; U.S. Pub. No. 2009/0198325; and U.S. Pub. No. 2011/0218623, the disclosures of which are incorporated by reference herein. For example, a 1-3 mm diameter inlay can be positioned under a flap or within a pocket of corneal tissue to increase the steepness of a central region of the corneal to increase near vision and provide distance vision in a region of the cornea peripheral to the central region. Other shapes can be used to correct other optical imperfections as well.

Keratophakia can be performed using artificial materials such as hydrogels, which are less biocompatible than corneal tissue. Alternative materials for corneal implants could help eliminate some of the potential issues with artificial materials.

### SUMMARY

One example of the disclosure is a corneal implant, such as an inlay or an onlay, having a body with an outermost diameter between about 1 and about 7 mm, the body comprising cultured corneal tissue. In some instances the corneal implant is meniscus shaped. In some instances the meniscus shape has a central thickness less than about 50 microns. In some instances the outermost diameter is between about 1 and about 3 mm. In some instances the corneal implant has an annular configuration, wherein the outermost diameter is the outermost diameter of the annular configuration.

One example of the disclosure is a method of manufacturing a corneal implant comprising creating a corneal implant from fabricated corneal tissue. Creating a corneal implant from fabricated corneal tissue can include cutting a corneal implant from fabricated corneal tissue using a laser.

One example of the disclosure is a corneal implant, such as an inlay or an onlay, having a body with an outermost diameter between about 1 and about 7 mm, the body constructed from living human tissue, such as a refractive lenticule extraction procedure. In some instances the corneal implant is meniscus shaped. In some instances the meniscus shape has a central thickness less than about 50 microns. In some instances the outermost diameter is between about 1 and about 3 mm. In some instances the corneal implant has an annular configuration, wherein the outermost diameter is the outermost diameter of the annular configuration.

One example of the disclosure is a method of manufacturing a corneal implant comprising creating a corneal implant from living corneal tissue.

One example of the disclosure is a method of manufacturing a corneal implant, comprising manufacturing a volume of corneal tissue; cutting the volume into a meniscus shaped lens with a 1-7 mm, optionally 1-3 mm diameter. Manufacturing the volume of corneal tissue optionally includes manufacturing the volume from human keratocytes. Manufacturing the volume from human keratocytes optionally comprises manufacturing the volume on a template. Manufacturing the volume from human keratocytes optionally comprises manufacturing the volume of corneal tissue to have aligned collagen fibrils.

One example of the disclosure is a corneal implant having a body with an outermost diameter of 1-7 mm, the body comprising manufactured corneal tissue. Optionally, the corneal implant has a meniscus-shaped body and a 1-3 mm diameter, the body comprising manufactured corneal tissue. Optionally the manufactured corneal tissue is made from human keratocytes. Optionally the manufactured corneal tissue has aligned collagen fibrils. Optionally the body has a central thickness of 50 microns or less.

### DETAILED DESCRIPTION

The disclosure is related to corneal implants and their methods of manufacture. “Corneal implants,” as the term is used herein, includes corneal inlays and corneal onlays. Corneal inlays are currently made from plastic-based materials such as hydrogels, which are less biocompatible than corneal tissue. A corneal reaction, such as keratocyte activity around the inlay, can result in local haziness.

An alternative material from which to construct inlays is artificial cornea, whether from a donor cornea or from fabricated corneal tissue such as in Gouveia. An inlay made of artificial cornea could be constructed using a femtosecond laser, similar to those employed to make corneal cuts for LASIK, which could cut the volume of the lens with sufficient accuracy. Alternatively, the inlay could be constructed by placing it on a lathe and cutting it down to the required size and configuration in the same manner as a contact lens is manufactured. This could also be done in a very cold environment in which the tissue is frozen for ease of cutting.

Such an inlay could then be placed under a kerototomy flap in such a way as to correct a patient's vision by amending the optical pathway of the eye by altering the surface of the cornea, examples of which are provided in U.S. Pat. No. 8,057,541, issued Nov. 15, 2011; U.S. Pub. No 2008/0262610, published Oct. 23, 2008; U.S. Pub. No 2009/0198325; and U.S. Pub. No. 2011/0218623.

The artificial cornea tissue can be used to construct inlays that can be used to correct vision in, for example, hyperopes, presbyopes, myopes and pseudophakic patients.

One of the advantages of implanting corneal implants made from the fabricated corneal tissue is that it solves a problem of corneal rejection of material introduced into it due to the biological nature of the inlay material. Corneal transplants have shown that the cornea is immune privileged, and as such a particular type of corneal tissue is not required for a particular individual. This is unlike, for instance, a blood transfusion where the right type of blood is required based on the individual's blood type. Small pieces of artificial cornea implanted within a cornea should therefore be well tolerated.

One aspect of the disclosure is a corneal implant, such as a corneal inlay, that has been manufactured from fabricated bio-prosthetic human corneal tissue. Another aspect of the disclosure is a method of constructing a corneal implant, such as a corneal inlay, from fabricated human corneal tissue.

The size and configurations of the corneal implants herein can be selected, or chosen, take into account epithelial remodeling of the cornea after implantations, which is described in U.S. Pat. No. 8,900,296, and which is incorporated herein by reference.

This disclosure also includes corneal implants, such as the inlays herein, which are constructed from transplanted human corneal tissue. In some aspect the corneal implants are constructed from corneal tissue that has been removed from living corneal tissue (i.e., removed while the subject is still alive). For example, the corneal implants can be constructed from living corneal tissue that has been removed from a subject's cornea during a refractive lenticule extraction (ReLEx) procedure, which includes femtosecond lenticule extraction (FLEx) and small incision lenticular extraction (SMILE) procedures. Cornea Lenticule Viability and Structural Integrity after Refractive Lenticule Extraction (ReLEx) and Cryopreservation (Mohamed-Noriega, Molecular Vision 2011; 17:3437-3449), which is incorporated herein by reference, describes exemplary techniques and procedures for obtaining and/or processing living corneal tissue from ReLEx procedures. Any of the methods and steps in Mohamed-Noriega can be used herein as part of the overall method of constructing the corneal implant. For example, a corneal lenticule removed from living corneal tissue during a SMILE procedure can be used to construct a corneal implant, such as a corneal inlay with a 1-4 mm diameter (such as 103 mm) for treating presbyopia.

In some embodiments the corneal implants are constructed from corneal tissue removed from recently deceased (e.g., within 24, or 48 hours after death) subjects. For example, any suitable volume of corneal tissue from a recently deceased individual can be removed and used to construct any of the corneal implants herein.

1-8. (canceled)

9. A method of manufacturing a corneal device, comprising
providing a volume of corneal tissue;
cutting the volume into a meniscus shaped lens with a 1-3 mm diameter.

10. The method of claim 9, wherein the cutting step comprises cutting the volume into a meniscus shaped lens with a laser.

11. The method of claim 9, wherein providing a volume of corneal tissue comprises manufacturing a volume of corneal tissue.

12. The method of claim 11, wherein manufacturing the volume of corneal tissue comprising manufacturing the volume from human keratocytes.

13. The method of claim 11, wherein manufacturing the volume from human keratocytes comprising manufacturing the volume on a template.

14. The method of claim 11, wherein manufacturing the volume from human keratocytes comprises manufacturing the volume of corneal tissue to have aligned collagen fibrils.

15. A corneal device, comprising:
a meniscus-shaped body with a 1-3 mm diameter, the body comprising corneal tissue.

16. The corneal device of claim 15, wherein the body comprises manufactured corneal tissue.

17. The corneal device of claim 16, wherein the manufactured corneal tissue is made from human keratocytes.

18. The corneal device of claim 16, wherein the manufactured corneal tissue has aligned collagen fibrils.

19. The corneal device of claim 15, wherein the body has a central thickness of 50 microns or less.
